(19) United States
(12) Patent Application Publication
(10) Pub. No.: US 2013/0190277 A1
(43) Pub. Date: Jul. 25, 2013

(54) Paraffin Wax Embedded Drug Delivery System

(71) Applicant: Joel Bain HERRON, Westerville, OH (US)

(72) Inventor: Joel Bain HERRON, Westerville, OH (US)

(21) Appl. No.: 13/719,789

(22) Filed: Dec. 19, 2012

Related U.S. Application Data
(60) Provisional application No. 61/590,749, filed on Jan. 25, 2012.

(51) Int. Cl. A61K 47/06; A61K 8/92

(52) U.S. Cl. CPC A61K 47/06; A61K 8/92
USPC 514/169; 514/789

(57) ABSTRACT

A paraffin wax embedded drug delivery system is a method for combining topical medications with paraffin wax for enhanced delivery and absorption of the topical medication at a treatment area on a human body. A quantity of paraffin wax and a topical medication are provided, and mixture measurements are specified for a desired concentration and a desired ratio of ingredients for the mixture. To combine the topical medication and the paraffin wax, the quantity of paraffin wax is heated, the topical medication is added to the liquid paraffin wax, the mixture is agitated to ensure even combination, and the mixture is cooled. To apply the mixture, the mixture is heated back to its liquid state and applied to a treatment area using a wax bath, a brush, a roller, or another method.

The current application claims a priority to the U.S. Provisional Patent application Ser. No. 61/590,749 filed on Jan. 25, 2012.

### FIELD OF THE INVENTION

The present invention relates generally to a method for delivering medication. More particularly, the present invention relates to a method for topical drug delivery by combining topical drugs with melted paraffin wax that may be applied directly to an individual's skin.

### BACKGROUND OF THE INVENTION

In chemistry, paraffin is a term that can be used synonymously with “alkane”, indicating hydrocarbons with the general formula CₙH₂ₙ₊₂. Paraffin wax refers to a mixture of alkanes that falls within the 20≦n≦40 range, which are found in the solid state at room temperature and begin to enter the liquid phase past approximately 37 degrees Centigrade *or 99 degrees Fahrenheit. Paraffin wax, or simply “paraffin”, is mostly found as a white, odorless, tasteless, waxy solid. Paraffin is an excellent material for storing heat, having a specific heat capacity of 2.14-2.9 joules per gram Kelvin and a heat fusion of 200-220 joules per gram. The heat retention properties of paraffin lend well to its use in therapy purposes.

Rehabilitation specialists, massage therapists, and the spa industry have widely endorsed paraffin therapy for its therapeutic properties. The use of paraffin wax therapy can be traced back to the Roman Empire, when massage therapists would pour hot waxes on their clients' bodies to prepare them for massages. Today, paraffin therapy is widely used to aid in the treatment of conditions such as arthritis, psoriasis, tendonitis, eczema, fibromyalgia, scar tissue, stiff joints, and more. Paraffin therapy is also useful in the treatment of sports related injuries and other conditions where heat therapy and exercise therapy are needed. Paraffin therapy also has the benefit that it soothes and moisturizes the skin, opens pores, improves circulation, improves range of motion, and promotes a sense of calm in the patient. Paraffin waxes are often offered as a part of many salon manicures and pedicures because it is relaxing, hydrating, and moisturizing to the skin, resulting in a silky, cosmetically-acceptable feeling to the skin.

A paraffin bath is the application of heated paraffin to a specific area of the body through the use of an open container of heated liquid paraffin. The body part is immersed in heated liquid paraffin wax and then withdrawn so that the wax solidifies to form an insulating layer. The procedure is repeated several times, and then the entire area is wrapped in an insulating material, such as a loose-fitting plastic bag or paper towels. This technique is effective for heating traumatized or inflamed areas, especially the hands, feet, and wrists, and is used primarily for patients with arthritis, rheumatism or other joint conditions.

Topical medication is a medication that is applied to body surfaces such as the skin or mucous membranes to treat ailments via a large range of classes including creams, foams, gels, lotions and ointments. Many topical medications are epicutaneous, meaning that they are applied directly to the skin. Topical medications may be embedded in a paraffin wax delivery method to combine the benefits of paraffin wax therapy with the effects of the medication. This method delivers warmth and hydration to skin as well as providing a water-resistant occlusive environment to enhance penetration of the topical medications. Occlusion of the area of application combined with increased warmth have the effect of increasing blood flow, resulting in greater penetration and absorption of the topical medications into the skin to enhance the desired effects of the paraffin embedded medications.

It is therefore an object of the present invention to provide a method by which a topical medication or multiple topical medications are combined with a paraffin wax delivery system and by which the topical medications and the melted paraffin wax are applied directly to the user's skin.

### BRIEF DESCRIPTION OF THE DRAWINGS

FIG. 1 is a flow chart illustrating the overall process of the present invention.

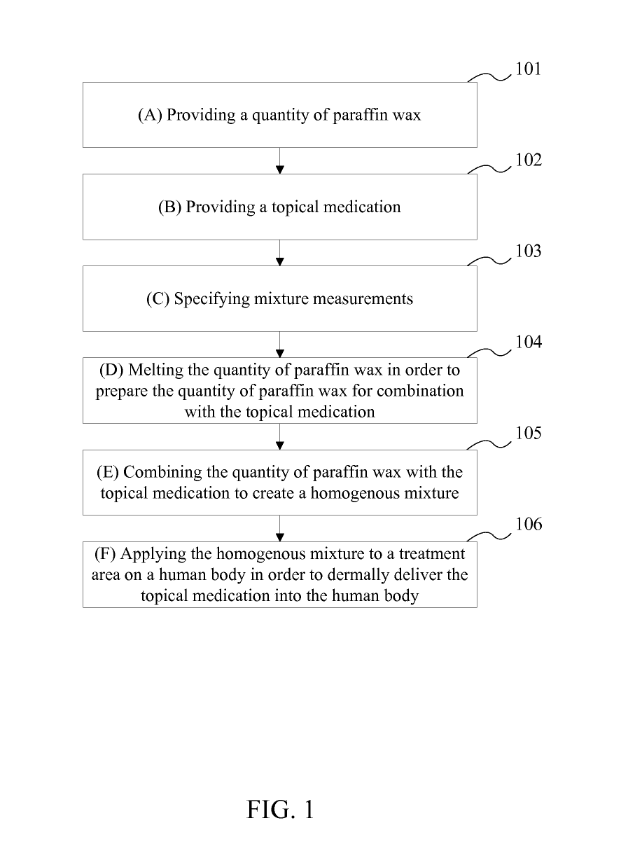

FIG. 2 is a flow chart illustrating the secondary process of the present invention for determining the concentration of the topical medication.

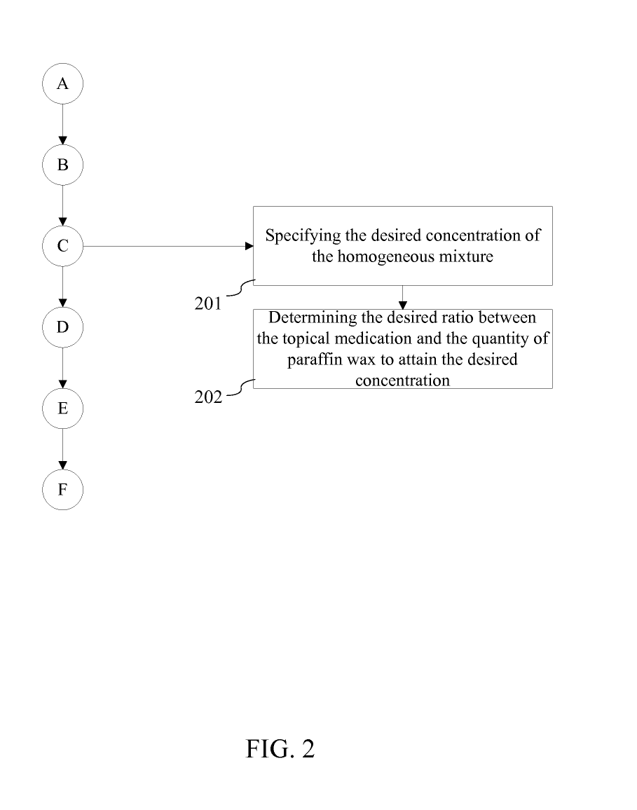

FIG. 3 is a flow chart illustrating the secondary processes of the present invention for combining the topical medication with the paraffin wax.

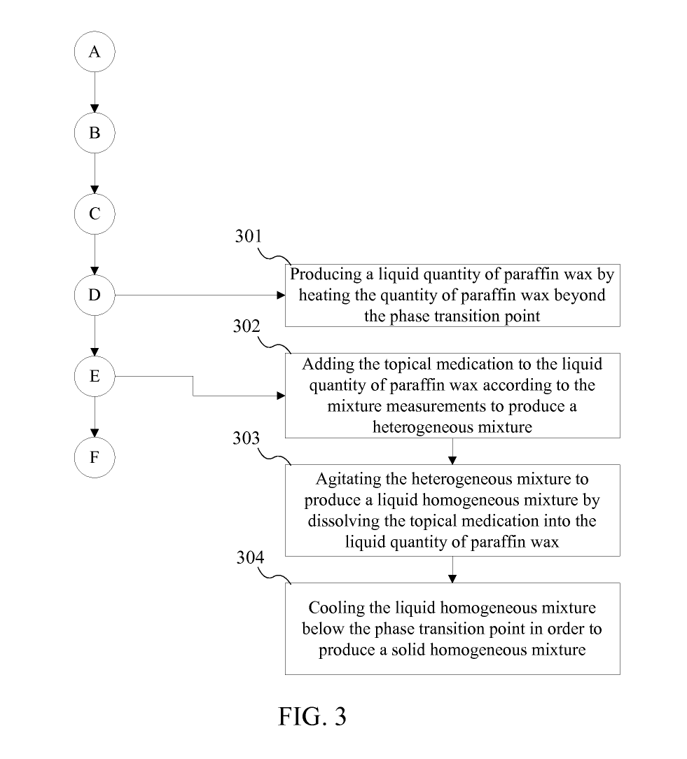

FIG. 4 is a flow chart illustrating the secondary processes of the present invention for applying the homogeneous mixture to a treatment area.

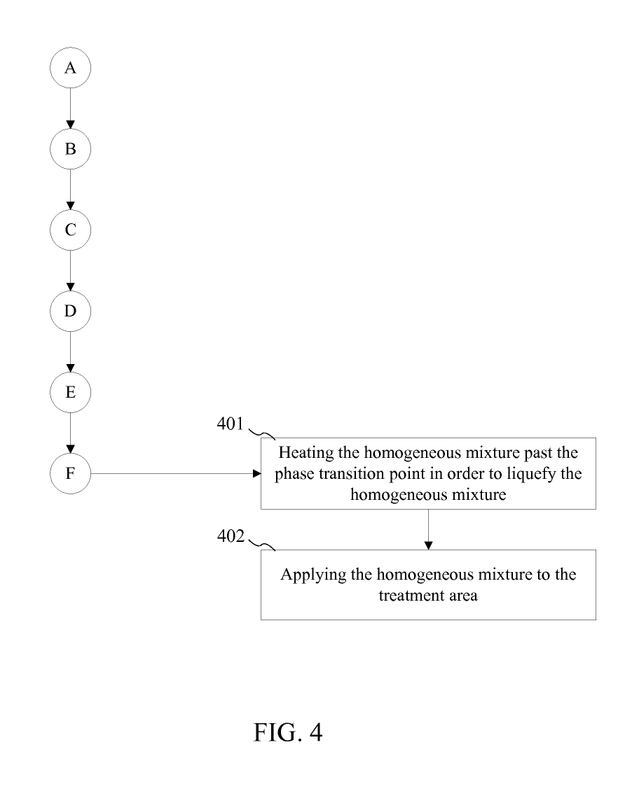

### DETAIL DESCRIPTIONS OF THE INVENTION

All illustrations of the drawings are for the purpose of describing selected versions of the present invention and are not intended to limit the scope of the present invention.

The present invention is a method for enhanced delivery of topical medications by embedding topical medications within paraffin wax to produce a homogenous mixture of the paraffin wax and the topical medications. In the preferred embodiment of the present invention, the topical medications are embedded within the paraffin wax during a manufacturing process. The manufacturing process to be utilized in the present invention is any appropriate process for producing individual saleable units of paraffin wax, particularly those already in existence. Well-known manufacturing methods for paraffin wax may be adapted for inclusion of topical medications. In another embodiment of the present invention, the topical medications are added to the paraffin wax by the consumer, whereby a user utilizes a commercially available wax bath to melt a quantity of paraffin wax, adds the topical medications, and stirs the wax bath in order to effectively combine the topical medications and the paraffin wax for use.

The topical medication to be utilized in the present invention may be any topical medication to be applied to a treatment area on a human body, wherein absorption of the topical medication into the treatment area is enhanced by combining the topical medication with the warming, moisturizing and occlusive effects of paraffin wax therapy. In one embodiment of the present invention, the topical medication is a topical steroid for treatment of conditions such as rash, eczema, psoriasis, palmoplantar pustulosis, and dermatitis, among other conditions. In another embodiment of the present invention, the topical medication is a topical analgesic such as a topical non-steroidal anti-inflammatory medication for treating conditions such as rheumatoid arthritis, arthralgia, osteoarthritis, and more. Other medications may be used in further alternate embodiments of the present invention, such as, but not limited to, cosmetic medications, anti-aging medications, urea for treating dry skin or hyperkeratosis, retinoids for skin rejuvenation or treatment of psoriasis, acne, or other skin conditions, acids, topical immunomodulators for various immunotherapy treatments, topical chemotherapy for pre-cancer treatment, and aminolevulinic acid for uses such as enhancing penetration of other medications through exposure to light.

In the preferred embodiment of the present invention, a quantity of paraffin wax is provided to be utilized with the method of the present invention 101. A topical medication is also provided to be utilized with the method of the present invention 102. Mixture measurements are specified for the quantity of paraffin wax and for the topical medication 103. The mixture measurements include characteristics such as, but not limited to, a desired concentration and a desired ratio for the homogeneous mixture. The desired concentration of the homogeneous mixture is specified to reflect an appropriate dosage for use of the topical medication 201. The desired ratio between the topical medication and the quantity of paraffin wax is determined to attain the desired concentration 202. In alternate embodiments of the present invention, further topical medications may be combined with the quantity of paraffin wax.

The quantity of paraffin wax is then combined with the topical medication to create a homogeneous mixture 105. The paraffin wax is melted in order to prepare the quantity of paraffin wax for combination with the topical medication 104. A liquid quantity of paraffin wax is produced by heating the quantity of paraffin wax beyond the phase transition point 301. To do this, heat is added to the quantity of paraffin wax until the quantity of paraffin wax reaches a liquid state. This may take place during the manufacturing process or in a wax bath utilized by the user. After the quantity of paraffin wax is transitioned to liquid, the topical medication is added to the liquid quantity of paraffin wax according to the mixture measurements in order to produce a heterogeneous mixture 302.

A liquid homogeneous mixture is then produced from the heterogeneous mixture. In the preferred embodiment of the present invention, the heterogeneous mixture is agitated by stirring in order to dissolve the topical medication into the liquid quantity of paraffin wax and produce the liquid homogeneous mixture 303. Additional or alternative steps to produce the liquid homogenous mixture may include, but are not limited to, pre-heating the topical medication to prepare the topical medication for combination with the quantity of paraffin wax, waiting a period of time for the topical medication to completely dissolve in the liquid quantity of paraffin wax, or adding another chemical to the mixture in order to facilitate the dissolution of the topical medication into the liquid quantity of paraffin wax. In an alternate embodiment of the present invention, the combination of the topical medication and the quantity of paraffin wax is not done by liquefying the quantity of paraffin wax, but is accomplished by some other method, such as, but not limited to, grinding the quantity of paraffin wax in its solid state into a powder, and adding the topical medication and shaking, stifling or applying pressure to the mixture. After the liquid homogeneous mixture is produced, the liquid homogeneous mixture is cooled below the phase transition point in order to solidify the homogeneous mixture 304. The solid homogeneous mixture may then be stored for later use, packaged, sold, shipped, and/or delivered to consumers or retailers for use or resale.

In the preferred embodiment of the present invention, the homogenous mixture is applied to the treatment area on the human body in order to dermally deliver the topical medication into the human body in an occlusive, water-resistant environment, increasing absorption of the topical medication applied to the treatment area and decreasing transepidermal water loss to the surrounding environment 106. The homogeneous mixture is again heated past the phase transition point in order to liquefy the homogeneous mixture 401, and the homogeneous mixture is applied to the treatment area 402. In the preferred embodiment of the present invention, the liquid homogeneous mixture is applied to the treatment area using a commercially available wax bath. In alternate embodiments of the present invention, the homogeneous mixture is applied to the treatment area using another method or apparatus, such as, but not limited to, painting the liquid homogeneous mixture onto the treatment area with a brush, applying the homogeneous mixture with a roller, a sheet, or applying the homogeneous mixture in solid form by rubbing or pressing a mass of solid homogeneous mixture onto the treatment area.

Although the invention has been explained in relation to its preferred embodiment, it is to be understood that many other possible modifications and variations can be made without departing from the spirit and scope of the invention as hereinafter claimed.

What is claimed is:

1. A method for a paraffin embedded drug delivery system comprises the steps of:
providing a quantity of paraffin wax, wherein the quantity of paraffin wax has a phase transition point;
providing a topical medication, wherein the topical medication acts by physically contacting human tissue surfaces;
specifying mixture measurements of the quantity of paraffin wax and of the topical medication, wherein the mixture measurements include a desired concentration and a desired ratio;
melting the quantity of paraffin wax in order to prepare the quantity of paraffin wax for combination with the topical medication;
combining the quantity of paraffin wax with the topical medication to create a homogenous mixture; and
applying the homogenous mixture to a treatment area on a human body, in order to dermally deliver the topical medication into the human body, wherein a water-resistant occlusive environment is created at the treatment area in order to enhance dermal delivery of the topical medication to the treatment area.

2. The method for a paraffin embedded drug delivery system as claimed in claim 1 comprises the steps of:
specifying the desired concentration of the homogenous mixture, wherein the desired concentration reflects an appropriate dosage for use of the topical medication; and
determining the desired ratio between the topical medication and the quantity of paraffin wax to attain the desired concentration.

3. The method for a paraffin embedded drug delivery system as claimed in claim 1 comprises the steps of:
producing a liquid quantity of paraffin wax by heating the quantity of paraffin wax beyond the phase transition point, wherein heat is added to the quantity of paraffin wax until the quantity of paraffin wax reaches a liquid state;
adding the topical medication to the liquid quantity of paraffin wax according to the mixture measurements to produce a heterogeneous mixture;
agitating the heterogeneous mixture in order to produce a liquid homogeneous mixture by dissolving the topical medication into the liquid quantity of paraffin wax; and
cooling the liquid homogenous mixture below the phase transition point in order to produce a solid homogenous mixture.

4. The method for a paraffin embedded drug delivery system as claimed in claim 1 comprises the steps of:
heating the homogenous mixture past the phase transition point in order to liquefy the homogenous mixture; and
applying the homogenous mixture to the treatment area, wherein the liquid homogenous mixture is applied using a wax bath, a brush, a roller, or a sheet, and wherein heat is delivered to the treatment area for enhanced blood flow and dermal penetration of the topical medication to the treatment area.

5. The method for a paraffin embedded drug delivery system as claimed in claim 1, wherein the topical medication is a topical steroid.

6. The method for a paraffin embedded drug delivery system as claimed in claim 1, wherein the topical medication is a topical analgesic.

7. The method for a paraffin embedded drug delivery system as claimed in claim 1, wherein the topical medication is a cosmetic medication.

8. The method for a paraffin embedded drug delivery system as claimed in claim 1, wherein further topical medications are combined with the quantity of paraffin wax.

9. A method for a paraffin embedded drug delivery system comprises the steps of:
providing a quantity of paraffin wax, wherein the quantity of paraffin wax has a phase transition point;
providing a topical medication, wherein the topical medication acts by physically contacting human tissue surfaces;
specifying mixture measurements of the quantity of paraffin wax and of the topical medication, wherein the mixture measurements include a desired concentration and a desired ratio;
melting the quantity of paraffin wax in order to prepare the quantity of paraffin wax for combination with the topical medication;
combining the quantity of paraffin wax with the topical medication to create a homogenous mixture;
applying the homogenous mixture to a treatment area on a human body, in order to dermally deliver the topical medication into the human body, wherein a water-resistant occlusive environment is created at the treatment area in order to enhance dermal delivery of the topical medication to the treatment area;
producing a liquid quantity of paraffin wax by heating the quantity of paraffin wax beyond the phase transition point, wherein heat is added to the quantity of paraffin wax until the quantity of paraffin wax reaches a liquid state;
adding the topical medication to the liquid quantity of paraffin wax according to the mixture measurements to produce a heterogeneous mixture;
agitating the heterogeneous mixture in order to produce a liquid homogeneous mixture by dissolving the topical medication into the liquid quantity of paraffin wax; and
cooling the liquid homogenous mixture below the phase transition point in order to produce a solid homogenous mixture.

10. The method for a paraffin embedded drug delivery system as claimed in claim 9 comprises the steps of:
specifying the desired concentration of the homogenous mixture, wherein the desired concentration reflects an appropriate dosage for use of the topical medication; and
determining the desired ratio between the topical medication and the quantity of paraffin wax to attain the desired concentration.

11. The method for a paraffin embedded drug delivery system as claimed in claim 9 comprises the steps of:
heating the homogenous mixture past the phase transition point in order to liquefy the homogenous mixture; and
applying the homogenous mixture to the treatment area, wherein the liquid homogenous mixture is applied using a wax bath, a brush, a roller, or a sheet, and wherein heat is delivered to the treatment area for enhanced blood flow and dermal penetration of the topical medication to the treatment area.

12. The method for a paraffin embedded drug delivery system as claimed in claim 9, wherein the topical medication is a topical steroid.

13. The method for a paraffin embedded drug delivery system as claimed in claim 9, wherein the topical medication is a topical analgesic.

14. The method for a paraffin embedded drug delivery system as claimed in claim 9, wherein the topical medication is a cosmetic medication.

15. The method for a paraffin embedded drug delivery system as claimed in claim 9, wherein further topical medications are combined with the quantity of paraffin wax.

16. A method for a paraffin embedded drug delivery system comprises the steps of:
providing a quantity of paraffin wax, wherein the quantity of paraffin wax has a phase transition point;
providing a topical medication, wherein the topical medication acts by physically contacting human tissue surfaces;
specifying mixture measurements of the quantity of paraffin wax and of the topical medication, wherein the mixture measurements include a desired concentration and a desired ratio;
melting the quantity of paraffin wax in order to prepare the quantity of paraffin wax for combination with the topical medication;
combining the quantity of paraffin wax with the topical medication to create a homogenous mixture;
applying the homogenous mixture to a treatment area on a human body, in order to dermally deliver the topical medication into the human body, wherein a water-resistant occlusive environment is created at the treatment area in order to enhance dermal delivery of the topical medication to the treatment area;
specifying the desired concentration of the homogenous mixture, wherein the desired concentration reflects an appropriate dosage for use of the topical medication;
determining the desired ratio between the topical medication and the quantity of paraffin wax to attain the desired concentration;
producing a liquid quantity of paraffin wax by heating the quantity of paraffin wax beyond the phase transition point, wherein heat is added to the quantity of paraffin wax until the quantity of paraffin wax reaches a liquid state;
adding the topical medication to the liquid quantity of paraffin wax according to the mixture measurements to produce a heterogeneous mixture;
agitating the heterogeneous mixture in order to produce a liquid homogeneous mixture by dissolving the topical medication into the liquid quantity of paraffin wax;
cooling the liquid homogenous mixture below the phase transition point in order to produce a solid homogenous mixture;
heating the homogenous mixture past the phase transition point in order to liquefy the homogenous mixture; and
applying the homogenous mixture to the treatment area, wherein the liquid homogenous mixture is applied using a wax bath, a brush, a roller, or a sheet, and wherein heat is delivered to the treatment area for enhanced blood flow and dermal penetration of the topical medication to the treatment area.

17. The method for a paraffin embedded drug delivery system as claimed in claim 16, wherein the topical medication is a topical steroid.

18. The method for a paraffin embedded drug delivery system as claimed in claim 16, wherein the topical medication is a topical analgesic.

19. The method for a paraffin embedded drug delivery system as claimed in claim 16, wherein the topical medication is a cosmetic medication.

20. The method for a paraffin embedded drug delivery system as claimed in claim 16, wherein further topical medications are combined with the quantity of paraffin wax.
